(19) United States
(12) Patent Application Publication
(10) Pub. No.: US 2005/0152851 A1
(43) Pub. Date: Jul. 14, 2005

(54) Semi-hard dentifrice

(76) Inventor: Basia Kaminski, Woodstock, NY (US)

Correspondence Address: JOHN MAIER, III, 666 AARON COURT, KINGSTON, NY, 12401, US

(21) Appl. No.: 10/757,168

(22) Filed: Jan. 13, 2004

(51) Int. Cl. A61K 7/16; A61K 7/20

(52) U.S. Cl. 424/49; 424/53

(57) ABSTRACT

The invention comprises a formulation for a semi-hard stick-type dentifrice optimally for use in an oral hygiene device which utilizes such semi-hard stick-type dentifrice, most often used when traveling. The dentifrice includes a primary base material such as glycerin. Sodium benzoate is included as a preservative. Sodium coco-sulfate and a mixture of wheat gluten proteins and potassium cocoate provide a foaming material. Cleansers are included and the dentifrice is strengthened by vegetal-based sodium stearate and vegetal-based stearyl alcohol. Whitening materials are included, as is an absorption material and sodium bicarbonate as an abrasive.

### BACKGROUND OF THE INVENTION

1. Field of the Invention

This invention relates to a formulation for a semi-hard oral hygiene dentifrice and, more specifically, to the formulation of a semi-hard hygiene dentifrice for travel purposes.

2. Prior Art and Objects

Oral hygiene agents such as toothpaste and mouthwash have long been well known in the art. Many of these oral hygiene agents are readily available to the ordinary consumer over the counter in tubes and canisters. Others are available by prescription. Still others are available only to dental professionals. Those toothpastes which are available to the public are generally slow-flowing, semi-liquid pastes comprising cleansing, abrasive and optimally decay preventive compositions of varying purposes and efficacy, which are packaged in a manner convenient for use in the home.

When the consumer is traveling, however, the container can open accidentally, or be punctured, and leak, with a resulting inconvenient mess.

The Kaminski Patent, U.S. Pat. No. 6,290,417, discloses a semi-hard stick-type dentifrice for use with an oral hygiene device, such as a toothbrush. This dentifrice has the advantage of not being subject to the leakage and mess occasioned by the tube and canister containers, and is therefore a convenient dentifrice to have when traveling. The dentifrice disclosed therein is comprised of baking soda, calcium ion, calcium carbonate, sodium lauryl sulphate, carrageenin, flavoring (specifically, peppermint leaf oil), calcium ascorbate and sea salt all in approximately equal quantities, combined with glycerin, and allowed to dry. This formulation, while efficacious for cleaning the oral cavity, is not commercially viable for several reasons, including the lack of a preservative to prolong its shelf life, the lack of an ingredient to prevent the condensation of water on the surface of the product (an “anti-sweat” ingredient), and the lack of a stabilizing agent to prevent the stick from softening or breaking too easily when in use. A formulation which is commercially viable is needed to allow the benefits of the semi-hard dentifrice invention disclosed in the previously cited Kaminski Patent, U.S. Pat. No. 6,290,417, to become accessible to the public.

### SUMMARY OF THE INVENTION

The present invention discloses a commercially viable semi-hard dentifrice which contains a stabilizer to prevent the stick from softening or breaking too easily when in use, a preservative to extend the shelf life of the dentifrice, and an anti-sweat ingredient to prevent the formation of condensate on the surface of the dentifrice. Optionally the improved dentifrice may also include a substance which has been shown to be effective in cavity prevention, such as fluoride ion. These improvements provide for the requirements of commercial viability, thus enabling the public to receive the full benefit of the semi-hard dentifrice disclosed. The stabilizer used in the present embodiment of the invention is a combination of a wax such as beeswax or ceresin wax, stearyl alcohol and sodium stearate. Wax functions to increase the melting point of, and adds tensile strength to, the finished product. Stearyl alcohol and sodium stearate each function to add tensile strength to the finished product; they comprise the main materials to form the shape of the product, optimally the shape of a stick, cone or cylinder, and provide the unique tensile strength of the dentrifice. Optimally, both stearyl alcohol and sodium stearate are vegetal based.

### DESCRIPTION OF THE PREFERRED EMBODIMENT

The semi-hard dentifrice is not designed for continual use in the home but may be used in the hone a well as when traveling. Rather, it is designed to be used when traveling. Its semi-hard nature prevents the problem of the dentifrice leaking and soiling clothing and other objects that may be packed in a suitcase or other luggage along with it. The semi-hard dentifrice is designed to be rubbed directly on the teeth, then brushed with a standard-type toothbrush. This provides the user with foaming action and mild abrasion action which cleans the teeth. Optimally, the semi-hard dentifrice also contains a decay preventive substance which provides the user with protection against dental caries and cavities when used. The dentifrice is optimally formed as a substantially straight stick, though other shapes are also possible. In its stick form, the dentifrice can be fitted to an oral hygiene device such as that disclosed in U.S. Pat. No. 6,290,417.

Glycerin provides the base for the dentifrice. It provides for gentle cleansing of the oral cavity. Glycerith-26 and glycerith-31 are alternatives to glycerin. The sudsing agent optimally is sodium coco-sulfate combined with a mix of potassium cocoate and wheat gluten proteins and potassium cocoate. These are natural cleansers which are not known to to have the carcinogenic properties of sodium lauryl sulfate, which was used in U.S. Pat. No. 6,290,417.

A gentle abrasive action is provided optimally by sodium bicarbonate. Sodium bicarbonate provides efficacious effects in dentrifice preparations. Abrasion is also provided by sodium chloride or, in the alternative, sea salt, and by calcium carbonate, or, in the alternative, calcium ascorbate. Calcium ascorbate has the additional benefit of providing the nutritive effects of ascorbate, or Vitamin C. Its use, however, is optional in the formulation of the dentrifice. Stearyl alcohol and sodium stearate are used as agents to promote tensile strength. These factors stabilize the shape of the dentifrice and help prevent excessive breakage of the dentifrice's stick form. The use of sodium stearate and stearyl alcohol in a dentifrice formulation is not known in the art. Because most toothpastes are of a pasty consistency, the need for the tensile strength provided by sodium stearate and stearyl alcohol has previously not been required. However, sodium stearate and stearyl alcohol are necessary to the present formulation to allow the finished product to maintain its shape and resist breakage either during use or when packed for travel.

A non-toxic wax, preferably beeswax, is used to increase the melting temperature of the dentifrice. The wax also aids in stabilizing the shape of the dentifrice.

Sodium benzoate provides a preservative effect for ingestibles and toothpaste. It is added to the formulation for this effect.

Flavoring for the dentifrice is optionally provided by plant oils, such as peppermint or spearmint oil and/or by artificial sweeteners. Flavoring is not necessary for the efficacy of the formulation, but its use dramatically improves the user's experience when using the product.

Coloring for the dentifrice may be provided as desired by titanium dioxide, FD&C Blue No. 1 and FD&C Yellow No. 5. Coloring is not required for the efficacious use of the formulation, but it makes the dentifrice more pleasant for the user. Moisture absorption is provided by aluminum starch octenylsuccinate. This prevents the dentifrice from “sweating” with moisture condensation on the surface of the stick. Without the use of an anti-sweat ingredient, moisture would accumulate on the dentifrice and act to destabilize its shape and composition. Use of a moisture-absorbing agent is therefore necessary to enhance the shelf ife of the dentifrice. Talc and nylon are possible alternatives.

A sweetener is necessary for a desirable taste for the consumer. Monoammonium glycyrrhizinate is a preferred sweetener, but a variety of sweeteners may be used to achieve a satisfying taste. A whitening material is important for a suitable dentifrice. Two whiteners are preferred, namely titanium dioxide and calcium carbonate. Either titanium dioxide or calcium carbonate may be used separately from the other, or they may be combined in various combinations.

A small amount of sodium chloride is included to provide an additional cleansing agent.

A specific formula to produce a preferred dentifrice by weight-to-weight percentages, and a range of each ingredient by weight-to-weight percentages is set forth below.

| | | Range of Percentages |
|---|---|---|
| Ingredient | Percent by Weight | by Weight |
| Glycerin | 41.40 | 38.40-45.40 |
| Sodium benzoate | 0.10 | 0.07-0.13 |
| Sodium coco-sulfate | 1.00 | 0.70-1.30 |
| Sodium chloride | 0.40 | 0.28-0.52 |
| Beeswax (Cera alba) | 0.30 | 0.21-0.39 |
| Stearyl alcohol | 0.80 | 0.56-1.04 |
| Sodium stearate | 7.00 | 5.00-9.00 |
| Monoammonium | 0.50 | 0.35-0.65 |
| glycyrrhizinate | | |
| Titanium dioxide | 5.00 | 3.60-6.50 |
| Calcium carbonate | 19.00 | 15.00-24.00 |
| Aluminum starch | 14.00 | 11.00-17.00 |
| octenylsuccinate | | |
| Sodium bicarbonate | 3.00 | 2.10-3.90 |
| Sodium coco-sulfate with | 6.00 | 4.50-7.50 |
| wheat gluten proteins and | | |
| potassium cocoate | | |
| Mentha piperita leaf oil | 1.50 | 1.05-1.95 |
| Water and Blue No. 1 | QS | QS |
| Water and Yellow No. 5 | QS | QS |

The quantity “QS” means a small quantity too insignificant for a percentage by weight.

Cavity prevention is optionally provided through use of the fluoride ion.

It is to be understood that the descriptive matter is in all cases to be interpreted as merely illustrative of the principles of the invention, rather than as limiting the same in any way, since it is contemplated that various changes may be made in various elements to achieve like results without departing from the spirit of the invention or the scope of the appended claims.

1. A semi-hard dentifrice comprising:
a primary base material;
sodium benzoate as a preservative;
sodium coco-sulfate and a mixture of wheat gluten proteins and potassium cocoate as a foaming material;
cleansers;
strengthening materials to promote tensile strength including vegetal-based sodium stearate and vegetal based stearyl alcohol;
a whitening material to whiten the teeth;
an absorption material to absorb moisture that forms on the dentifrice; and sodium bicarbonate to provide a gentle abrasive action.

2. A semi-hard dentifrice according to claim 1 wherein the cleansers include sodium coco-sulfate.

3. A semi-hard dentifrice according to claim 1 wherein the cleansers include sodium chloride.

4. A semi-hard dentifrice according to claim 1 wherein the cleansers include sea salt.

5. A semi-hard dentifrice according to claim 1 wherein the primary base material is glycerin.

6. A semi-hard dentifrice according to claim 1 wherein the primary base material is glycerith-26.

7. A semi-hard dentifrice according to claim 1 wherein the primary base material is glycerith-31.

8. A semi-hard dentifrice according to claim 1 wherein the semi hard dentifrice further includes a flavoring.

9. A semi-hard dentifrice according to claim 1 wherein the semi-hard dentifrice further includes Mentha piperita leaf oil as flavoring.

10. A semi-hard dentifrice according to claim 1 wherein the semi-hard dentifrice further includes a coloring agent.

11. A semi-hard dentifrice comprising:
a primary base material in a percentage range by weight of 38.40 to 45.40;
sodium benzoate as a preservative in a percentage range by weight of 0.07 to 0.13;
sodium bicarbonate as a gentle abrasive in a percentage range by weight of 2.10 to 3.90;
calcium carbonate as a cleansing agent in a percentage range by weight of 15.00 to 24.00;
sodium coco-sulfate and a mixture of wheat gluten proteins and potassium cocoate as a foaming agent in a percentage range by weight of 4.50 to 7.50;
a wax in a percentage range by weight of 0.21 to 0.39;
strengthening materials to promote tensile strength including vegetal based sodium stearate in a percentage range by weight of 5.00 to 9.00 and vegetal based stearyl alcohol in a percentage range by weight of 0.56 to 1.04; and
cleansers in a percentage range by weight of 0.98 to 1.82.

12. A semi-hard dentifrice according to claim 11 wherein the cleansers include sodium coco-sulfate in a percentage range by weight of 0.70 to 1.30 and sodium chloride in a percentage range by weight of 0.28 to 0.52.

13. A semi-hard dentifrice according to claim 11 wherein the primary base material is glycerin.

14. A semi-hard dentifrice according to claim 11 wherein the primary base material is glycerith-26.

15. A semi-hard dentifrice according to claim 11 wherein the primary base material is glycerith-31.

16. A semi-hard dentifrice according to claim 11 wherein the semi-hard dentifrice further includes a flavoring.

17. A semi-hard dentifrice according to claim 11 wherein the semi-hard dentifrice further includes a coloring agent.

18. A semi-hard dentifrice comprising:
glycerin in a percentage by weight of 41.40;
sodium benzoate in a percentage by weight of 0.10;
sodium coco-sulfate in a percentage by weight of 1.00;
sodium chloride in a percentage by weight of 0.40;
beeswax in a percentage by weight of 0.30;
stearyl alcohol in a percentage by weight of 0.80;
sodium stearate in a percentage by weight of 7.00;
monoammonium glycyrrhizinate in a percentage by weight of 0.50;
titanium dioxide in a percentage by weight of 5.00;
calcium carbonate in a percentage by weight of 19.00;
aluminum starch octenylsuccinate in a percentage by weight of 14.00;
sodium bicarbonate in a percentage by weight of 3.00; and
wheat gluten proteins in a percentage by weight of 6.00.

19. A semi-hard dentifrice according to claim 18 wherein the semi-hard dentifrice further includes a coloring agent.

20. A semi-hard dentifrice according to claim 18 further including Mentha piperita leaf oil in a percentage by weight of 1.50.
